(19) United States
(12) Patent Application Publication
(10) Pub. No.: US 2009/0247804 A1
(43) Pub. Date: Oct. 1, 2009

(54) METHODS FOR CONVERSION OF METHANE TO USEFUL HYDROCARBONS AND CATALYSTS FOR USE THEREIN

(75) Inventors: Joe D. Sauer, Baton Rouge, LA (US); Tyson J. Hall, Baton Rouge, LA (US); George Wyndham Cook, Baton Rouge, LA (US); Michael J. Krause, Orchard Park, NY (US); Paul F. Ranken, Baton Rouge, LA (US)

Correspondence Address: ALBEMARLE CORPORATION;PATENT DEPARTMENT, 451 FLORIDA STREET, BATON ROUGE, LA, 70801, US

(73) Assignee: Albemarle Corporation, Baton Rouge, LA (US)

(21) Appl. No.: 12/442,226

(22) PCT Filed: Sep. 14, 2007
(86) PCT No.: PCT/US07/78489

§ 371 Date: Mar. 20, 2009

(30) Foreign Application Priority Data
Sep. 21, 2006 (US) 60846274

(51) Int. Cl. B01J 31/14; C07C 2/86

(52) U.S. Cl. 585/733; 502/103

(57) ABSTRACT

Methods are provided for converting methane to useful hydrocarbons. In the methods provided, a fluid comprising methane and hydrogen is combined with a catalyst composition derived from at least an aluminum compound, such as an aluminum halide, an aluminum alkyl, or a aluminum hydrate, and a second component such as a transition metal halide, a transition metal hydride, or a zero-valent metal, to produce heavier hydrocarbons.

### BACKGROUND

Methane is a major constituent of natural gas and also of biogas. World reserves of natural gas are constantly being upgraded. However, a significant portion of the world reserves of natural gas is in remote locations, where gas pipelines frequently cannot be economically justified. Natural gas is often co-produced with oil in remote offsite locations where reinjection of the gas is not feasible. Much of the natural gas produced along with oil at remote locations, as well as methane produced in petroleum refining and petrochemical processes, is flared. Since methane is classified as a greenhouse gas, future flaring of natural gas and methane may be prohibited or restricted. Thus, significant amounts of natural gas and methane are available to be utilized.

Different technologies have been described for utilizing these sources of natural gas and methane. For example, technologies are available for converting natural gas to liquids, which are more easily transported than gas. Various technologies are described for converting methane to higher hydrocarbons and aromatics.

The Fischer Tropsch reaction has been known for decades. It involves the synthesis of liquid (or gaseous) hydrocarbons or their oxygenated derivatives from the mixture of carbon monoxide and hydrogen (synthesis gas) obtained by passing steam over hot coal. This synthesis is carried out with metallic catalysts such as iron, cobalt, or nickel at high temperature and pressure. The overall efficiency of the Fischer Tropsch reaction and subsequent water gas shift chemistry is estimated at about 15%, and while it does provide a route for the liquefication of coal stocks, it is not adequate in its present level of understanding and production for conversion of methane-rich stocks to liquid fuels.

It is possible to hydrogenate carbon monoxide to generate methanol. Methanol, by strict definition of the “gas to liquid” descriptor, would seem to fulfill the target desire of liquefication of normally gaseous, toxic feedstocks. However, in many regards, the oxygen containing molecules have already relinquished a significant percentage of their chemical energy by the formation of the C—O bond present. A true “methane to liquid hydrocarbon” process would afford end products that would not suffer these losses.

Yet another approach for methane utilization involves the halogenation of the hydrocarbon molecule to halomethane and subsequent reactions of that intermediate in the production of a variety of materials. Again, the efficiency and overall cost performance of such routes would be commercially prohibitive. Such a halogenation process would also suffer from the decrease of stored chemical energy during the C—X bond formation. Additionally, the halogen species has to be satisfactorily accounted for (i.e., either recycled, or captured in some innocuous safe form) within the end-use of the product from this overall route.

Gas to liquid processes that can convert methane into liquid fuels have been a significant challenge to the petrochemical industry at large. Of note are the works of Karl Ziegler and Giulio Natta regarding aluminum catalysts for ethylene chain growth, culminating in the 1963 Nobel Prize for Chemistry; the work of George Olah in carbocation technology, for which Mr. Olah received the 1994 Nobel Prize for Chemistry; and the work of Peter Wasserscheid regarding transition metal catalysis in ionic liquid media.

In spite of technologies that are currently described and available, a need exists for commercially feasible means for converting methane to useful hydrocarbons.

### THE INVENTION

This invention meets the above-described need by providing catalyst compositions useful for converting methane to C₅ and higher hydrocarbons, which catalyst compositions are derived from (or prepared by combining) at least (i) AlHₙX¹ₘRₚ, where Al is aluminum, H is hydrogen, each X¹ is a halogen and can be the same as, or different-from, any other X¹, each R is a, C₁ to C₄ alkyl and can be the same as, or different from, any other R, each of n and m is independently 0, 1, or 2, and p is 1 or 2, all such that (n+m+p)=3, and (ii) M^{v}H_{q}X²ᵣ, where M^{v} is a metal of valence v, H is hydrogen, each X² is a halogen and can be the same as, or different from, any other X², and each of q and r is 0 or any integer through and including v, all such that (q+r)=v. The valence of M^{v}, (i.e., v) can be zero. This invention includes catalyst compositions derived from (or prepared by combining) at least two or more of such AlHₙX¹ₘRₚ, where each AlHₙX¹ₘRₚ can be the same as, or different from, any other AlHₙX¹ₘRₚ and two or more of such M^{v}H_{q}X²ᵣ, where each M^{v}H_{q}X², can be the same as, or different from, any other M^{v}H_{q}X²ᵣ. Additionally, this invention includes catalyst compositions derived from (or prepared by combining) at least AlHₙXₘRₚ where either n or m is zero, and M^{v}H_{q}X²ᵣ, where M^{v} is a metal of valence v, H is hydrogen, each X² is a halogen and can be the same as, or different from, any other X², and each of q and r is 0 or any integer through and including v, all such that (q+r)=v. Catalyst compositions according to this invention are also useful for converting methane and C₂ to C₄ alkanes to C₅ and higher hydrocarbons.

This invention also provides methods comprising combining at least (i) a fluid comprising H₂ and methane, (ii) AlHₙX¹ₘRₚ, where Al is aluminum, H is hydrogen, each X¹ is a halogen and can be the same as, or different from, any other X¹, each R is a C₁ to C₄ alkyl and can be the same as, or different from, any other R, each of n and m is independently 0, 1, or 2₁ and p is 1 or 2, all such that (n+m+p)=3, and (iii) M^{v}H_{q}X²ᵣ, where M^{v} is a metal of valence v, H is hydrogen, each X² is a halogen and can be the same as, or different from, any other X², and each of q and r is 0 or any integer through and including v, all such that (q+r)=v; and producing C₅ and higher hydrocarbons. This invention also provides methods comprising combining at least (i) a fluid comprising H₂ and methane and either (ii) two or more of such AlHₙX¹ₘRₚ, where each AlHₙX¹ₘRₚ can be the same as, or different from, any other AlHₙX¹ₘRₚ and/or two or more of such M^{v}H_{q}X², where each M^{v}H_{q}X²ᵣ can be the same as, or different from, any other M^{v}H_{q}X²ᵣ; (ii) AlHₙX¹ₘRₚ where either of n or m is zero; and producing C₅ and higher hydrocarbons.

### AlHₙX¹ₘRₚ

Suitable compounds AlHₙX¹ₘRₚ include, for example, aluminum methyl chloride (AlMeCl₂), aluminum methyl bromide (AlMeBr₂), mono-chloro aluminum methyl hydride (AlHMeCl) and mono-bromo aluminum methyl hydride (AlHMeBr). Other suitable compounds AlHₙX¹ₘRₚ are known or may come to be known, as will be familiar to those skilled in the art and having the benefit of the teachings of this invention.

### Transition Metal Halides and Related Compounds M^{v}H_{q}X²ᵣ

Suitable transition metal halides and related compounds M^{v}H_{q}X²ᵣ can be derived from components comprising transition metals such as titanium and vanadium and from components comprising halogen atoms such as chlorine, bromine, iodine, etc. For example, titanium bromide (TiBr₄) is a suitable transition metal halide. Suitable transition metal halides M^{v}H_{q}X²ᵣ include, for example, TiX²₃ (“titanium haloform”) where q is zero and each X² is a halogen atom (such as chlorine or bromine) and can be the same as, or different from, any other X². Other suitable transition metal halides and related compounds M^{v}H_{q}X²ᵣ are known or may come to be known, as will be familiar to those skilled in the art and having the benefit of the teachings of this invention.

### Transition Metal Hydrides and Related Compounds M^{v}H_{q}X²ᵣ

Suitable transition metal hydrides and related compounds M^{v}H_{q}X²ᵣ can be derived from components comprising transition metals such as titanium and vanadium and from components comprising hydrogen atoms. For example, titanium hydride (TiH₄) is a suitable transition metal hydride. Other suitable transition metal hydrides and related compounds M^{v}H_{q}X²ᵣ are known or may come to be known, as will be familiar to those skilled in the art and having the benefit of the teachings of this invention.

### Zero-Valent Metals

Suitable zero-valent metals include, for example, any metal with at least one, electron in its outermost (non-S) shell or with at least one electron more than d⁵ or f⁷ levels. Suitable zero-valent metals include Ti⁰, Al⁰, and Zr⁰. Numerous suitable zero-valent metals are known or may come to be known as will be familiar to those skilled in the art and having the benefit of the teachings of this invention.

This invention provides that the metal halide component can allow for the methane conversion to take place in a essentially liquid state at modest operating parameters (e.g., temperatures of about 200° C. and pressures at or below about 200 atmospheres).

his invention provides methods of converting methane to useful hydrocarbons by facilitating polymerization of methane substantially without the normally required conversion to an oxidized species, such as carbon monoxide. According to this invention, methane is converted to useful hydrocarbons via a substantially direct catalytic process.

Methane can be converted, in the presence of catalyst compositions according to this invention and/or according to methods of this invention, to a reactive species capable of combining with other methane (or heavier products obtained from earlier reaction of this species) molecules to give carbon-carbon bond formation in an efficient manner, without substantial conversion to carbon/coke/charcoal by products. This activation also takes place in such fashion that oxidation of methane to carbon monoxide (such as seen in Fischer-Tropsch and water gas shift reactions) is not required and does not occur in substantial amounts. The products of the technology of this invention would be highly branched, highly methylated hydrocarbons such as those desired for high octane gasoline fuel stocks.

Without limiting this invention, the following compounds may be formed in situ when catalyst compositions according to this invention and/or methods according to this invention are used: M^{v}H.2(AlX²₂), M^{v}H₂.2(AlHX²), M^{v}X².2(AlX²₂₂), and M^{v}X²₂.2(AlX²₂); also the following where M is M^{v} as defined herein and X can be either an X¹ or an X² as defined herein:

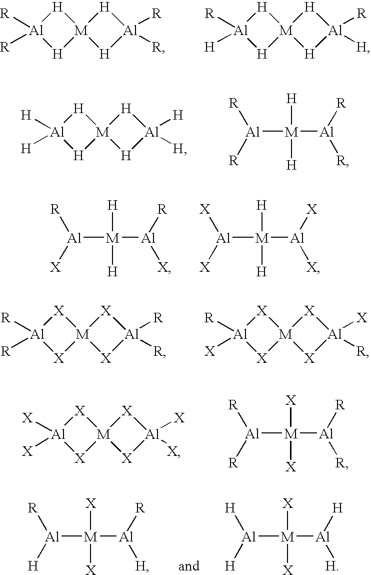

This invention allows for the conversion of the under-utilized, and heretofore difficult to modify, hydrocarbon feed-stock methane in the generation of various higher hydrocarbons. The product hydrocarbons can be used as liquid fuels. This is not limiting, in that many of the higher hydrocarbons (chemical products) produced by methods of this invention could have value in excess of that of gasoline or diesel liquid fuel stocks.

Use of this invention could amount to substantial revenues in a refinery—where the technology could be applied—when using methane in place of the normal crude oil feedstocks. Additionally, if the technology can be adapted to small, remote, independent operations (such as found on drilling and production platforms remote from pipeline service) the profits would be amplified dramatically, since the natural gas in produced is such remote locations is typically flared.

Use of this invention can also be applied to the production of higher value-added chemical stocks for use as intermediates in many chemical manufacturing processes, or as the final chemical product itself.

Another advantage of the use of methods of this invention is the production of elemental hydrogen as a co-product to the hydrocarbon fraction. One mole of H₂ is liberated for every mole of methanol converted to methane. The produced hydrogen could be utilized as valuable, pollution-free fuel. Additionally, it could be utilized as a raw material or reactant in any of manifold applications in chemical production requiring a hydrogen source for reduction, hydrogenation, and so forth. Hydrogen is used in many industrial activities such as the manufacture of fertilizers, petroleum processing, methanol synthesis, annealing of metals and producing electronics materials. In the foreseeable future, the emergence of fuel cell technology may extend the use of hydrogen to domestic and vehicle applications.

It is to be understood that the reactants and components referred to anywhere in the specification or claims hereof, whether by chemical name or formula or otherwise, and whether referred to in the singular or plural, are identified as they exist prior to coming into contact with another substance (e.g., another component, a solvent, etc.). It matters not what chemical changes, transformations and/or reactions, if any, take place in the resulting mixture or solution as such changes, transformations and/or reactions are the natural result of bringing the specified components together under the conditions specified. Thus the components are identified as ingredients to be brought together in performing a desired operation or in forming a desired composition. Also, even though the claims may refer to substances, components and/or ingredients in the present tense (“comprises”, “is”, etc.), the reference is to the substance, component or ingredient as it existed at the time just before it was first contacted, blended or mixed with one or more other substances, components and/or ingredients in accordance with the present disclosure and the claim thereof. As will be familiar to those skilled in the art, the terms “combined” and “combining” as used herein mean that the components that are “combined” or that one is “combining” are put into a container with each other.

While the present invention has been described in terms of one or more preferred embodiments, it is to be understood that other modifications may be made without departing from the scope of the invention, which is set forth in the claims below.

We claim:

1. A catalyst composition useful for converting methane to C5 and higher hydrocarbons, which catalyst composition is derived from at least (i) AlHₙX¹ₘRₚ, where Al is aluminum, H is hydrogen, each X¹ is a halogen and can be the same as, or different from, any other X¹, each R is a C₁ to C₄ alkyl and can be the same as, or different from, any other R, each of n and m is independently 0, 1 or 2, and p is 1 or 2, all such that (n+m+p)=3, and (ii) M^{v}H_{q}X²ᵣ, where M^{v} is a metal of valence v, H is hydrogen, each X² is a halogen and can be the same as, or different from, any other X², and each of q and r is 0 or any integer through and including v, all such that (q+r)=v.

2. A catalyst composition according to claim 1 wherein the AlHₙX¹ₘRₚ comprises aluminum methyl bromide.

3. A catalyst composition according to claim 1 wherein the M^{v}H_{q}X²ᵣ comprises titanium bromide.

4. A catalyst composition useful for converting C₁ to C₄ alkanes to C5 and higher hydrocarbons, which catalyst composition is derived from at least Al and M^{v}H_{q}X²ᵣ, where Al is aluminum, M^{v} is a metal of valence v, H is hydrogen, each X² is a halogen and can be the tame as, or different from, any other X², and each of q and r is 0 or any integer through and including v, all such that (q+r)=v.

5. A method comprising combining at least (i) a fluid comprising H₂ and methane, (ii) AlHₙX¹ₘRₚ, where Al is aluminum, H is hydrogen, each X¹ is a halogen and can be the same as, or different from, any other X¹, each R is a C₁ to C₄ alkyl and can be the same as, or different from, any other R, each of n and m is 0, 1, or 2, and p is 1 or 2, all such that (n+m+p)=3, and (iii) M^{v}H_{q}X²ᵣ, where M^{v} is a metal of valence v, H is hydrogen, each X² is a halogen and can be the same as, or different from, any other X², and each of q and r is 0 or any integer through and including v, all such that (q+r)=v, and producing C5 and higher hydrocarbons.
